(19) United States
(12) Patent Application Publication
(10) Pub. No.: US 2025/0107513 A1
(43) Pub. Date: Apr. 3, 2025

(54) METHOD FOR COUNTING EMBRYOS DEVELOPING IN A UTERUS OF CAENORHABDITIS ELEGANS

(71) Applicant: Shanghai University, SHANGHAI (CN)

(72) Inventors: Hui LI, SHANGHAI (CN); Chen WANG, SHANGHAI (CN); Xiaoli WANG, SHANGHAI (CN); Chongli SHI, SHANGHAI (CN); Yeyong LI, SHANGHAI (CN); Lingjun ZENG, SHANGHAI (CN); Yi PENG, SHANGHAI (CN)

(73) Assignee: Shanghai University, SHANGHAI (CN)

(21) Appl. No.: 18/973,133

(22) Filed: Dec. 9, 2024

Related U.S. Application Data
(63) Continuation of application No. PCT/CN2022/140214, filed on Dec. 20, 2022.

(30) Foreign Application Priority Data
Jun. 9, 2022 (CN) 202210650976.8

(51) Int. Cl. A01K 67/30; C12N 1/20; C12R 1/19; G01N 33/50

(52) U.S. Cl. CPC A01K 67/30; C12N 1/20; G01N 33/5005; A01K 2227/703; C12R 2001/19

(57) ABSTRACT

In a method for counting embryos developing in a uterus of Caenorhabditis elegans, a complete set of experimental procedures are designed to count embryos in the uterus of Caenorhabditis elegans by using Caenorhabditis elegans as a model organism. The method is applicable to Caenorhabditis elegans exposed from an L1 stage to a final stage of pregnancy, and can provide an easy-to-observe, fast, simple, accurate and reliable experimental method to apply Caenorhabditis elegans to research of reproductive ability, detection results of which are highly accurate and reliable, and the experimental method is suitable for promotion.

### CROSS-REFERENCE TO RELATED APPLICATIONS

The present application is a continuation-application of International (PCT) Patent Application No. PCT/CN2022/140214, filed on Dec. 20, 2022, which claims priority to Chinese Patent Application No. 202210650976.8, filed on Jun. 9, 2022 and entitled “METHOD FOR COUNTING EMBRYOS DEVELOPING IN UTERUS OF CAENORHABDITIS ELEGANS”, the entire contents of which are hereby incorporated by reference in their entirety.

### FIELD OF THE INVENTION

The present invention pertains to the field of biotechnologies, and specifically, to a method for counting embryos developing in a uterus of Caenorhabditis elegans.

### BACKGROUND

As a non-parasitic nematode, Caenorhabditis elegans is an ideal model organism for research since it has virtues of ease of culture, translucent body, short life cycle, and complete genome sequencing database. In most cases, Caenorhabditis elegans is hermaphroditic and has a complete reproductive structure, including two gonadal arms, two seminal vesicles, and a uterus, which enable it to reproduce through autogamy. Therefore, Caenorhabditis elegans is a classic model for the study of reproductive effects in disciplines such as biomedicine and environmental toxicology. Generally, related physiological research indexes for studying reproduction of Caenorhabditis elegans include gonadal apoptosis, genital morphology, the number of embryos developing in a uterus, morphology and the number of oocytes, morphology and the number of sperms, the number of laid eggs, an egg hatching rate, and so on.

Because completely developed gonads, uterus, and intestine of Caenorhabditis elegans shield some embryos in the nematode during pregnancy, the embryos in the pregnant nematode are inaccurately counted under direct observation. In addition, because Caenorhabditis elegans creeps rapidly under the microscope, it is difficult to clearly determine morphology and the number of embryos developing in the uterus of the nematode through observation, and it is impossible to count embryos in the uterus of each nematode, thereby causing large errors and inaccurate results in evaluation of the reproductive effects. Therefore, it is necessary to record the morphology or the number of embryos after further transparent treatment of pregnant Caenorhabditis elegans in a short period of time without affecting the morphology and quality of the embryos.

A normal growth cycle of hatched eggs of Caenorhabditis elegans includes L1, L2, L3, L4, juvenile and adult stages. Generally, exposure factor treatment of Caenorhabditis elegans starts from the L1 stage, and the embryos are counted during pregnancy (that is, embryos at the adult stage in the uterus of Caenorhabditis elegans are observed under the microscope). Therefore, based on the foregoing conditions, an experimental procedure for counting embryos developing in the uterus of Caenorhabditis elegans is designed.

### SUMMARY

In view of existing problems in the prior art, the present invention provides a method for counting embryos developing in a uterus of Caenorhabditis elegans. In the present invention, NaClO solution is mainly used to pretreat Caenorhabditis elegans, to dissolve macromolecules of proteins in bodies of pregnant nematodes by virtue of strong oxidation of NaClO, so that the bodies of Caenorhabditis elegans become transparent or even completely dissolved without destroying structures and morphology of eggs in a short period of time, which completely releases and exposes the embryos developing in the uteri of adult nematodes, thereby facilitating morphology observation and counting of the embryos under a stereomicroscope. The method of present invention has an advantage of short detection cycle and simple and rapid operation process, and the method effectively improves accuracy and reliability of the counting of embryos in the uterus in reproductive indexes of Caenorhabditis elegans.

A technical solution of the present invention provides a method for counting embryos developing in a uterus of Caenorhabditis elegans, specifically including the following steps:
step 1: preparation of an E. coli solution: selecting freshly prepared E. coli OP50 as feed for Caenorhabditis elegans, and according to intended use, dividing the E. coli solution into two parts, where one part of the E. coli solution is used to coat an NGM medium and the other part is used to feed Caenorhabditis elegans daily when exposed to triphenyl phosphate (TPHP);
where in step 1, the process for preparing the NGM medium is as follows: adding 3 g of NaCl, 17 g of agar, 2.5 g of peptone, 0.111 g of CaCl₂ and 0.24 g of MgSO₄ to 1 L of deionized water, stirring a resulting mixture for dissolution, sterilizing the mixture at 121° C. for 25 minutes, and after sterilization, cooling the medium to 55° C. to 60° C., adding 1 mL of cholesterol solution and 25 mL of potassium phosphate buffer solution, stirring a resulting mixture for even mixing, and loading 15 mL of medium onto a 7 cm petri dish with a peristaltic pump, to obtain the NGM medium after cooling and solidification at room temperature;
where the 5 mg/mL cholesterol solution is prepared with absolute ethanol, and a method for preparing the potassium phosphate buffer solution is as follows: adding 10.83 g of KH₂PO₄ and 4.7 g of K₂HPO₄ to each 100 mL of ultrapure water, stirring a resulting mixture for dissolution, and sterilizing the mixture at 121° C. for 25 minutes;
step 2: coating of E. coli: drawing an appropriate amount of E. coli for coating onto a surface of the NGM medium with a pipette, then applying E. coli at the center of the petri dish evenly with a sterilized coating rod or bottom of a test tube, and after E. coli is completely attached to the NGM medium and forms a thin film, placing the petri dish upside down in a 20° C. dark biochemical incubator for later use;
step 3: culture of Caenorhabditis elegans: inoculating an appropriate number of Caenorhabditis elegans on the surface of the NGM medium containing E. coli, observing a growth status of Caenorhabditis elegans daily, and if E. coli is consumed out completely, replacing the medium in a timely manner until most Caenorhabditis elegans are pregnant;
step 4: lysis of Caenorhabditis elegans: rinsing pregnant Caenorhabditis elegans under culture from a petri dish into a 1.5 mL centrifuge tube with K solution 3 times, discarding a supernatant after centrifugation, adding 1 mL of lysis solution into the centrifuge tube, subjecting a resulting mixture to vortex oscillation vigorously for 1.5 minutes, then discarding a supernatant after centrifugation again, adding the lysis solution, subjecting the resulting mixture to vortex oscillation vigorously until the pregnant nematodes are completely lysed and the eggs are completely exposed, collecting the eggs and washing the eggs (of Caenorhabditis elegans) 3 to 5 times to remove the residual lysis solution;
step 5: synchronization treatment of Caenorhabditis elegans: adding nematode eggs dropwise to the NGM medium containing no E. coli, culturing the nematode eggs at 20° C. for 16 to 20 hours under dark conditions, where Caenorhabditis elegans are in the L1 stage, and rinsing Caenorhabditis elegans at the stage into a 1.5 mL centrifuge tube with the K solution 3 times;
step 6: preparation of exposure solutions: weighing 0.1000 g of TPHP solid accurately, dissolving the TPHP solid in dimethyl sulfoxide until a concentration reaches 10 μg/L, then diluting TPHP solutions with dimethyl sulfoxide again until concentrations reach 10 mg/L, 100 mg/L, 1000 mg/L and 5000 mg/L to be used as mother liquors later; using dimethyl sulfoxide as the mother liquor of the blank control group, further diluting the mother liquors by 100 folds with the solution and then diluting the diluted mother liquors by 100 folds with a K+ solution to finally obtain exposure solutions of Caenorhabditis elegans containing 0 μg/L, 1 μg/L, 10 μg/L, 100 μg/L and 500 μg/L TPHP.
step 7: exposure of Caenorhabditis elegans to a TPHP solution: adding a prepared TPHP exposure solution to a sterile 6-well plate with 5 mL of prepared TPHP exposure solution per well, then evenly adding Caenorhabditis elegans at the L1 stage to the 6-well plate after the synchronization treatment, feeding Caenorhabditis elegans with 100 μg/L E. coli daily, and culturing Caenorhabditis elegans for 72 hours;
step 8: selection of Caenorhabditis elegans at an exposure stage: after exposure to TPHP, transferring Caenorhabditis elegans to the NGM medium containing E. coli OP50, culturing Caenorhabditis elegans until pregnancy, then transferring pregnant Caenorhabditis elegans to a 1.5 mL centrifuge tube and washing the pregnant Caenorhabditis elegans with the K solution 3 times; step 9: preparation of NaClO solution: taking NaClO with effective chlorine content of 5.5% to 6.5% and diluting NaClO at a volume ratio of 1:4 (NaClO:K solution) to obtain an experimental NaClO solution for later use;
step 10: transparent treatment of Caenorhabditis elegans: adding the prepared NaClO solution to a sterile 24-well plate with 1 mL of the prepared NaClO solution per well, then adding 10 to 20 Caenorhabditis elegans to each well, and after 5 minutes of standing at room temperature, observing states of Caenorhabditis elegans every 2 minutes until nematode bodies are transparent;
step 11: counting of embryos in the uterus of Caenorhabditis elegans: putting the 24-well plate containing transparent Caenorhabditis elegans under an optical stereomicroscope, and counting exposed embryos of each Caenorhabditis elegans; and
step 12: data analysis: analyzing data by using SPSS software, setting a confidence interval of samples to 95%, and calculating an intra-group mean and mean error of the numbers of embryos developing in nematodes in different treatment groups.

Further, in step 1,
the first manner for preparing E. coli for coating the NGM medium is to inoculate monoclonal E. coli into an LB culture medium, centrifuge monoclonal E. coli at 37° C. at 150 rpm and incubate the monoclonal E. coli for 16 hours to 20 hours under darkness; and
the second manner for preparing E. coli for feeding Caenorhabditis elegans daily is to centrifuge the first E. coli solution at 12000 rpm for 10 minutes, where E. coli is then at a bottom of a centrifuge tube; and discard the supernatant and replace the original supernatant with the K solution at a ratio of 1:1 (original supernatant:K solution (v:v)).

Further, in step 1, the process for preparing the K solution medium includes adding 2.386 g of KCl and 2.98 g of NaCl to each 1 L of ultrapure water, stirring a resulting mixture for even mixing, and sterilizing the resulting mixture at 121° C. for 25 minutes.

In step 1, the method for preparing the LB culture medium includes adding 10 g of peptone, 5 g of yeast powder and 5 g of NaCl to each 1 L of ultrapure water, stirring a resulting mixture for dissolution, and sterilizing the resulting mixture at 121° C. for 25 minutes.

Further, in step 3, the process for changing the medium includes: rinsing the original Caenorhabditis elegans on the surface of the medium into a 1.5 mL centrifuge tube with the K solution 3 times, centrifuging the centrifuge tube to remove the supernatant, and then adding Caenorhabditis elegans at the bottom dropwise to the new medium with E. coli.

Further, in step 1 and step 4, the process for preparing the lysis solution includes: adding 2.5 mL of NaClO solution with effective chlorine content of 5.5% to 6.5%, 7.5 mL of K solution and 0.1 g of NaOH to each 10 mL of lysis solution.

Further, in step 6, the process for preparing the K+ solution includes: adding 2.386 g of KCl, 2.98 g of NaCl, 0.333 g of CaCl₂, and 0.36 g of MgSO₄ to each 1 L of ultrapure water, stirring a resulting mixture for even mixing, sterilizing the resulting mixture at 121° C. for 20 minutes, and adding 1 mL of cholesterol solution after cooling, where the K+ solution is prepared immediately before use.

As compared with the prior art, the present invention has the following beneficial effects:

First, chemicals and medicines soluble in water or soluble in water under solubilization of dimethyl sulfoxide are generally selected in prior art as exposure factors, but exposure treatment of the method in the present invention is performed in a liquid, so that the exposure solution of Caenorhabditis elegans is at a uniform concentration.

Second, experimental conditions are highly applicable. In the present invention, only termination time of exposure is limited, that is, Caenorhabditis elegans need to be in the pregnancy stage when embryos in their uteri are counted; and a starting point of exposure is not limited. The present invention can be applied to the following experimental conditions:
(1) The treatment of exposure factors starts from the L1 stage and ends until pregnancy.
(2) The treatment of exposure factors starts from the L1 stage, and ends at a non-pregnant stage, and Caenorhabditis elegans are transferred to the NGM medium coated with E. coli OP50 and further cultured until pregnancy.
(3) The treatment of exposure factors starts between the L2 stage and the L4 stage and ends until pregnancy.
(4) The treatment of exposure factors starts between the L2 stage and the L4 stage, and ends at a non-pregnant stage, and Caenorhabditis elegans are transferred to the NGM medium coated with E. coli OP50 and further cultured until pregnancy.

Third, the NaClO solution is used for concentration research in the experiments in the present invention, and is easy to prepare. At this experimental concentration, the pregnant nematodes can be quickly transparentized or even dissolved, while the embryos can maintain their original morphology for at least 1 hour. In the present invention, the NaClO solution is added before Caenorhabditis elegans, which can fix the nematodes at the center during addition. If Caenorhabditis elegans are added before the NaClO solution, the nematodes are flushed to an edge of the plate by the liquid, which is not conducive to observation and counting. In the present invention, a 24-well plate is selected for experimental observation. On the one hand, the 24-well plate is highly transparent. On the other hand, the added NaClO solution can be prevented from overflowing, and a field of view of the stereomicroscope can exactly completely cover each well of the 24-well plate, which avoids repeated counting.

### BRIEF DESCRIPTION OF DRAWINGS

FIG. 1 is a flowchart of a method in the present invention;

FIG. 2 is a schematic diagram of the number of embryos developing in Caenorhabditis elegans treated in the method in the present invention; and

FIG. 3 is a statistical diagram of the number of embryos in uteri of Caenorhabditis elegans that are exposed to TPHP and that are treated in the method in the present invention.

### DETAILED DESCRIPTION OF EMBODIMENTS

To more clearly describe the technical solutions and advantages of the present invention, the present invention is further described in detail below with reference to the accompanying drawings and the specific embodiments.

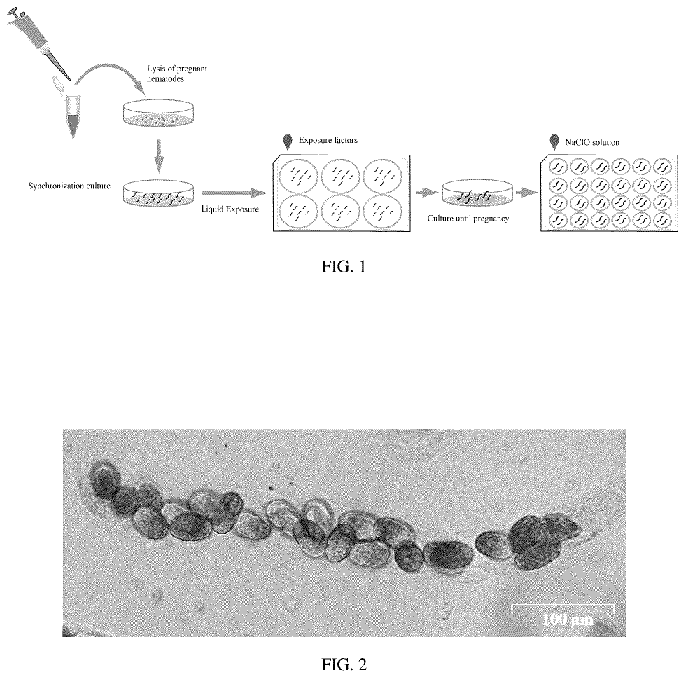

As shown in a flowchart (FIG. 1) of a method in the present invention, this embodiment provides a method for counting embryos developing in a uterus of Caenorhabditis elegans exposed to a chemical of triphenyl phosphate (TPHP) for 72 hours, specifically including the following steps:

Step 1: Prepare an E. coli solution. Freshly prepared E. coli OP50 is selected as feed for Caenorhabditis elegans in the experiment. According to intended use, the E. coli solution can be divided into two parts, where one part of the E. coli solution is used to coat an NGM medium and the other part is used to feed Caenorhabditis elegans daily when exposed to TPHP. The first method for preparing E. coli for coating the NGM medium is to inoculate monoclonal E. coli into an LB culture medium, centrifuge monoclonal E. coli at 37° C. at 150 rpm and incubate the monoclonal E. coli for 16 hours to 20 hours under darkness. The second method for preparing E. coli for feeding Caenorhabditis elegans daily is to centrifuge the first E. coli solution at 12000 rpm for 10 minutes, where E. coli is then at a bottom of a centrifuge tube; and discard the supernatant and replace the original supernatant with the K solution at a ratio of 1:1 (original supernatant:K solution (v:v)).

In step 1, a method for preparing the NGM medium includes: adding 3 g of NaCl, 17 g of agar, 2.5 g of peptone, 0.111 g of CaCl₂ and 0.24 g of MgSO₄ to 1 L of deionized water, stirring a resulting mixture for dissolution, and sterilizing the mixture at 121° C. for 25 minutes; and after sterilization, cooling the medium to 55° C. to 60° C., adding 1 mL of cholesterol solution and 25 mL of potassium phosphate buffer solution, stirring a resulting mixture for even mixing, and loading 15 mL of medium onto a 7 cm petri dish with a peristaltic pump, to obtain the NGM medium after cooling and solidification at room temperature. The 5 mg/mL cholesterol solution is prepared with absolute ethanol and a method for preparing the potassium phosphate buffer solution is as follows: adding 10.83 g of KH₂PO₄ and 4.7 g of K₂HPO₄ to each 100 mL of ultrapure water, stirring a resulting mixture for dissolution, and sterilizing the mixture at 121° C. for 25 minutes.

In step 1, the method for preparing the K solution medium is to add 2.386 g of KCl and 2.98 g of NaCl to each 1 L of ultrapure water, stir a resulting mixture for even mixing, and sterilize the resulting mixture at 121° C. for 25 minutes.

In step 1, the method for preparing the LB culture medium is to add 10 g of peptone, 5 g of yeast powder and 5 g of NaCl to each 1 L of ultrapure water, stir a resulting mixture for dissolution, and sterilize the resulting mixture at 121° C. for 25 minutes.

Step 2: Apply E. coli. An appropriate amount of E. coli for coating is drawn onto a surface of the NGM medium with a pipette, and then E. coli is applied at the center of the petri dish evenly with a sterilized coating rod or bottom of a test tube. It should be noted that an edge of the colony is about 1 cm away from a wall of the petri dish to prevent Caenorhabditis elegans from crawling from an edge of the petri dish to the bottom or the side wall. After E. coli is completely attached to the NGM medium and forms a thin film, the petri dish is placed upside down in a 20° C. dark biochemical incubator for later use.

Step 3: Culture Caenorhabditis elegans. An appropriate number of Caenorhabditis elegans are inoculated on the surface of the NGM medium with E. coli, a growth status of Caenorhabditis elegans is observed daily, and if E. coli is consumed completely, the medium is replaced in a timely manner until most Caenorhabditis elegans are pregnant (embryos in the uterus of the nematode can be seen under a stereomicroscope). A method for changing the medium is as follows: rinsing the original Caenorhabditis elegans on the surface of the medium into a 1.5 mL centrifuge tube with the K solution 3 times, centrifuge the centrifuge tube to remove the supernatant, and then add Caenorhabditis elegans at the bottom dropwise to the new medium with E. coli.

Step 4: Lyse Caenorhabditis elegans. Pregnant Caenorhabditis elegans under culture are rinsed from a petri dish into a 1.5 mL centrifuge tube with K solution 3 times, and a supernatant is discarded after centrifugation. 1 mL of lysis solution is added into the centrifuge tube and a resulting mixture is subjected to vortex oscillation vigorously for 1.5 minutes. Then a supernatant is discarded after centrifugation again, the lysis solution is added, and the resulting mixture is subjected to vortex oscillation vigorously until the pregnant nematodes are completely lysed and the eggs are completely exposed. The eggs are collected and the eggs are washed 3 to 5 times to remove the residual lysis solution.

In step 4, a method for preparing the lysis solution is as follows: adding 2.5 mL of NaClO solution with effective chlorine content of 5.5% to 6.5%, 7.5 mL of K solution and 0.1 g of NaOH to each 10 mL of lysis solution.

Step 5: Subject Caenorhabditis elegans to synchronization treatment. Nematode eggs are added dropwise to the NGM medium containing no E. coli, the nematode eggs are cultured at 20° C. for 16 to 20 hours under dark conditions, where Caenorhabditis elegans are in the L1 stage, and Caenorhabditis elegans at the stage are rinsed into a 1.5 mL centrifuge tube with the K solution 3 times.

Step 6: Prepare exposure solutions. 0.1000 g of TPHP solid is weighed accurately, the TPHP solid is dissolved in dimethyl sulfoxide until a concentration reaches 10 g/L, and then TPHP solutions are diluted with dimethyl sulfoxide again until concentrations reach 10 mg/L, 100 mg/L, 1000 mg/L and 5000 mg/L to be used as mother liquors later. Dimethyl sulfoxide is used as the mother liquor of the blank control group. The mother liquors are further diluted by 100 folds with the K solution and then diluting the diluted mother liquors by 100 folds with a K+ solution to finally obtain exposure solutions of Caenorhabditis elegans containing 0 μg/L (control group), 1 μg/L, 10 μg/L, 100 μg/L and 500 μg/L TPHP.

In step 6, a method for preparing the K+ solution is as follows: adding 2.386 g of KCl, 2.98 g of NaCl, 0.333 g of CaCl₂, and 0.36 g of MgSO₄ to each 1 L of ultrapure water, stirring a resulting mixture for even mixing, sterilizing the resulting mixture at 121° C. for 20 minutes, and adding 1 mL of cholesterol solution after cooling, where the K+ solution is prepared immediately before use.

Step 7: Expose Caenorhabditis elegans to a TPHP solution. A prepared TPHP exposure solution is added to a sterile 6-well plate with 5 mL of prepared TPHP exposure solution per well. Caenorhabditis elegans at the L1 stage are then evenly added to the 6-well plate after the synchronization treatment, Caenorhabditis elegans are fed with 100 μg/L E. coli daily, and Caenorhabditis elegans are cultured for 72 hours.

Step 8: Select Caenorhabditis elegans at an exposure stage. The method in the present invention is applicable to pregnant Caenorhabditis elegans, and this is because Caenorhabditis elegans are no longer pregnant after being exposed to TPHP for 72 hours. After exposure to TPHP, Caenorhabditis elegans are transferred to the NGM medium containing E. coli OP50, Caenorhabditis elegans are cultured until pregnancy, then pregnant Caenorhabditis elegans are transferred to a 1.5 mL centrifuge tube and the pregnant Caenorhabditis elegans are washed with the K solution 3 times to be used for the experimental research.

Step 9: Prepare NaClO solution. In the present invention, NaClO with effective chlorine content of 5.5% to 6.5% is taken and NaClO is diluted at a volume ratio of 1:4 (NaClO:K solution) to obtain an experimental NaClO solution for later use.

Step 10: Subject Caenorhabditis elegans to transparent treatment. The prepared NaClO solution is added to a sterile 24-well plate with 1 mL of prepared NaClO solution per well. Then 10 to 20 Caenorhabditis elegans are added to each well, and after 5 minutes of standing at room temperature, states of Caenorhabditis elegans are observed every 2 minutes until nematode bodies are transparent and embryos developing in the uteri of adult nematodes are all exposed. As shown in FIG. 2, the state can last for at least one hour. Therefore, in this method, it is recommended to complete the counting of embryos in the uterus within 1 hour.

Step 11: Count embryos in the uterus of Caenorhabditis elegans. The 24-well plate containing transparent Caenorhabditis elegans is put under an optical stereomicroscope, and exposed embryos of each Caenorhabditis elegans are counted, where exposed embryos of at least 25 nematodes are counted in each treatment group.

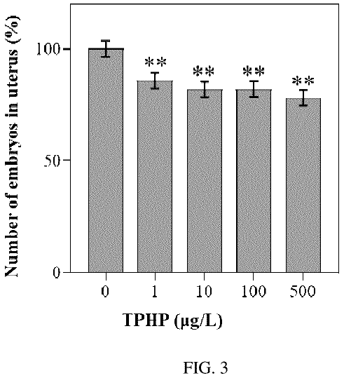

Step 12: Analyze data. Data is analyzed by using SPSS software, a confidence interval of samples is set to 95%, and an intra-group mean and mean error of the numbers of embryos developing in nematodes in different treatment groups are calculated, where the data can be selectively in a format of a mean percentage or multiple based on requirements of the experiment. For a comparison method, a control group can also be compared with the test group and different test groups can also be compared with each other according to requirements of the experiment. Specifically, as shown in FIG. 3, GraphPad Prism is used for drawing.

Description of results: In the method of the present invention, Caenorhabditis elegans are exposed to TPHP and then transferred to a petri dish for culture until pregnancy; and as shown in FIG. 2, after transparent treatment of pregnant nematodes with NaClO, a schematic diagram of morphology and the number of embryos in the uteri of the pregnant nematodes is obtained, the schematic diagram shows a total of 22 embryos, the morphology of the embryos is fully shown and the embryos are explicit to count. During the research process, the embryos in the uterus of each pregnant nematode exposed to TPHP at different concentrations are further counted, at least 25 pregnant nematodes are exposed to each group of TPHP at the concentration, SPSS is used for statistical analysis, one-way analysis of variance and Dunnett's post-hoc test are used for inter-group comparison between different concentration groups and the control group, and the data is denoted as a percentage of the mean±standard error relative to the control group, where **p<0.01, indicating a statistically significant difference between TPHP exposure groups and the control group. As shown in FIG. 3, under exposure to TPHP at concentrations of 1 μg/L, 10 μg/L, 100 μg/L and 500 μg/L, the numbers of embryos in the uteri decrease by 14.2%, 18.2%, 18.1% and 21.8% respectively, where p<0.01, and the numbers of embryos are significantly different, indicating that TPHP exposure can significantly reduce the number of embryos developing in nematodes and TPHP has a potential reproductive toxicity, thereby providing basic data support for further research on the reproductive toxicity effects and specific molecular mechanisms of such chemicals.

Although the present invention is disclosed in detail with reference to the accompanying drawings, it should be understood that these descriptions are merely exemplary but are not intended to limit the application of the present invention. The protection scope of the present invention is defined by the appended claims, and may include various variations, modifications, and equivalent solutions made to the invention without departing from the protection scope and spirit of the present invention.

1. A method for counting embryos developing in a uterus of Caenorhabditis elegans, comprising the following steps:
step 1: preparation of an E. coli solution: selecting freshly prepared E. coli OP50 as feed for Caenorhabditis elegans, which is divided into two parts according to its intended use, wherein one part of the E. coli solution is used to coat an NGM medium and the other part is used to feed Caenorhabditis elegans daily when exposed to triphenyl phosphate, which is name as TPHP;
wherein preparing process the NGM medium in step 1 is as follows: adding 3 g of NaCl, 17 g of agar, 2.5 g of peptone, 0.111 g of CaCl₂ and 0.24 g of MgSO₄ to 1 L of deionized water, stirring a resulted mixture for dissolution, sterilizing the mixture at 121° C. for 25 minutes, and after sterilization, cooling the medium to 55° C. to 60° C., adding 1 mL of cholesterol solution and 25 mL of potassium phosphate buffer solution, stirring the mixture for even mixing, and loading 15 mL of medium onto a 7 cm petri dish with a peristaltic pump, to obtain the NGM medium after cooling and solidification at room temperature;
wherein the 5 mg/mL cholesterol solution is prepared with absolute ethanol and the process for preparing the potassium phosphate buffer solution is as follows: adding 10.83 g of KH₂PO₄ and 4.7 g of K₂HPO₄ to each 100 mL of ultrapure water, stirring a resulted mixture for dissolution, and sterilizing the mixture at 121° C. for 25 minutes;
step 2: coating of E. coli: drawing an appropriate amount of E. coli for coating onto a surface of the NGM medium with a pipette, then coating E. coli at the center of the petri dish evenly with a sterilized coating rod or bottom of a test tube, and after E. coli is completely attached to the NGM medium and forms a thin film, placing the petri dish upside down in a 20° C. dark biochemical incubator for later use;
step 3: culture of Caenorhabditis elegans: inoculating an appropriate number of Caenorhabditis elegans on the surface of the NGM medium with E. coli, observing a growth status of Caenorhabditis elegans daily, and if E. coli is consumed completely, replacing the medium in a timely manner until most Caenorhabditis elegans are pregnant;
step 4: lysis of Caenorhabditis elegans: rinsing pregnant Caenorhabditis elegans under culture from a petri dish into a 1.5 mL centrifuge tube with K solution 3 times, discarding a supernatant after centrifugation, adding 1 mL of lysis solution into the centrifuge tube, subjecting a resulted mixture to vortex oscillation vigorously for 1.5 minutes, then discarding a supernatant after centrifugation again, adding the lysis solution, subjecting the resulting mixture to vortex oscillation vigorously until the pregnant nematodes are completely lysed and the eggs are completely exposed, collecting the eggs and washing the eggs 3 to 5 times to remove the residual lysis solution;
step 5: synchronization treatment of Caenorhabditis elegans: adding nematode eggs dropwise to the NGM medium containing no E. coli, culturing the nematode eggs at 20° C. for 16 to 20 hours under dark conditions, at this time, Caenorhabditis elegans are in the L1 stage, and rinsing Caenorhabditis elegans at the stage into a 1.5 mL centrifuge tube with the K solution 3 times;
step 6: preparation of exposure solutions: weighing 0.1000 g of TPHP solid accurately, dissolving the TPHP solid in dimethyl sulfoxide until a concentration reaches 10 g/L, then diluting TPHP solutions with dimethyl sulfoxide again until concentrations reach 10 mg/L, 100 mg/L, 1000 mg/L and 5000 mg/L to be used as mother liquors later, using dimethyl sulfoxide as the mother liquor of the blank control group, further diluting the mother liquors by 100 folds with the K solution and then diluting the diluted mother liquors by 100 folds with a K+ solution to finally obtain exposure solutions of Caenorhabditis elegans containing 0 μg/L, 1 μg/L, 10 μg/L, 100 μg/L and 500 μg/L TPHP.
step 7: exposure of Caenorhabditis elegans to a TPHP solution: adding a prepared TPHP exposure solution to a sterile 6-well plate with 5 mL of prepared TPHP exposure solution per well, then evenly adding Caenorhabditis elegans at the L1 stage to the 6-well plate after the synchronization treatment, feeding Caenorhabditis elegans with 100 μg/L E. coli daily, and culturing Caenorhabditis elegans for 72 hours;
step 8: selection of Caenorhabditis elegans at an exposure stage: after exposure to TPHP, transferring Caenorhabditis elegans to the NGM medium containing E. coli OP50, culturing Caenorhabditis elegans until pregnancy, then transferring pregnant Caenorhabditis elegans to a 1.5 mL centrifuge tube and washing the pregnant Caenorhabditis elegans with the K solution 3 times;
step 9: preparation of NaClO solution: taking NaClO with effective chlorine content of 5.5% to 6.5% and diluting NaClO at a volume ratio of 1:4 (NaClO:K solution) to obtain an experimental NaClO solution for later use;
step 10: transparent treatment of Caenorhabditis elegans: adding the prepared NaClO solution to a sterile 24-well plate with 1 mL of the prepared NaClO solution per well, then adding 10 to 20 Caenorhabditis elegans to each well, and after 5 minutes of standing at room temperature, observing states of Caenorhabditis elegans every 2 minutes until nematode bodies are transparent;
step 11: counting of embryos in the uterus of Caenorhabditis elegans: putting the 24-well plate containing transparent Caenorhabditis elegans under an optical stereomicroscope, and counting exposed embryos of each Caenorhabditis elegans; and
step 12: data analysis: analyzing data by using SPSS software, setting a confidence interval of samples to 95%, and calculating an intra-group mean and mean error of the numbers of embryos developing in nematodes in different treatment groups.

2. The method for counting embryos developing in a uterus of Caenorhabditis elegans according to claim 1, wherein in step 1, process of preparing E. coli can adopt one of below manners:
(1) the first manner for preparing E. coli for coating the NGM medium is to inoculate monoclonal E. coli into an LB culture medium, centrifuge monoclonal E. coli at 37° C. at 150 rpm and incubate the monoclonal E. coli for 16 hours to 20 hours under darkness; and
(2) the second manner for preparing E. coli for feeding Caenorhabditis elegans daily is to centrifuge the E. coli solution obtained by the first manner (1) at 12000 rpm for 10 minutes, wherein E. coli is then at a bottom of a centrifuge tube; and discard the supernatant and replace the original supernatant with the K solution at a ratio of 1:1 (original supernatant:K solution (v:v)).

3. The method for counting embryos developing in a uterus of Caenorhabditis elegans according to claim 1, wherein,
the process of preparing the K solution medium comprises: adding 2.386 g of KCl and 2.98 g of NaCl to each 1 L of ultrapure water, stirring a resulting mixture for even mixing, and sterilizing the resulting mixture at 121° C. for 25 minutes.

4. The method for counting embryos developing in a uterus of Caenorhabditis elegans according to claim 2, wherein,
the process of preparing the LB culture medium is includes: adding 10 g of peptone, 5 g of yeast powder and 5 g of NaCl to each 1 L of ultrapure water, stirring a resulting mixture for dissolution, and sterilizing the resulting mixture at 121° C. for 25 minutes.

5. The method for counting embryos developing in a uterus of Caenorhabditis elegans according to claim 1, wherein in step 3, the process of changing the medium is as follows: rinsing the original Caenorhabditis elegans on the surface of the medium into a 1.5 mL centrifuge tube with the K solution 3 times, centrifuging the centrifuge tube to remove the supernatant, and then adding Caenorhabditis elegans at the bottom dropwise to the new medium with E. coli.

6. The method for counting embryos developing in a uterus of Caenorhabditis elegans according to claim 1, wherein in step 4, the process of preparing the lysis solution is as follows: adding 2.5 mL of NaClO solution with effective chlorine content of 5.5% to 6.5%, 7.5 mL of K solution and 0.1 g of NaOH to each 10 mL of lysis solution.

7. The method for counting embryos developing in a uterus of Caenorhabditis elegans according to claim 1, wherein in step 6, the process of preparing the K+ solution is as follows: adding 2.386 g of KCl, 2.98 g of NaCl, 0.333 g of CaCl₂, and 0.36 g of MgSO₄ to each 1 L of ultrapure water, stirring a resulting mixture for even mixing, sterilizing the resulting mixture at 121° C. for 20 minutes, and adding 1 mL of cholesterol solution after cooling, wherein the K+ solution is prepared immediately before use.
